(19) United States
(12) Patent Application Publication
(10) Pub. No.: US 2004/0127993 A1
(43) Pub. Date: Jul. 1, 2004

(54) Spreader implant for placement between vertebrae

(76) Inventors: Erich Kast, Pfungen (CH); Hans-Joachim Wilke, Dornstadt (DE); Peter Weiland, Nonnweiler-Braunshausen (DE)

Correspondence Address: Friedrich Kueffner, Suite 910, 317 Madison Avenue, New York, NY, 10017, US

(21) Appl. No.: 10/686,037

(22) Filed: Oct. 15, 2003

(30) Foreign Application Priority Data
Oct. 16, 2002 (DE) 102 48 171.7

(51) Int. Cl. A61F 2/44

(52) U.S. Cl. 623/17.16; 623/17.11

(57) ABSTRACT

An implant for placement between vertebrae, especially as used for joining vertebrae after a diskectomy. The shape of the implant is adapted to a depression present in the vertebral surfaces facing the implant. This adaptation to the slightly depressed vertebral surface results in the implant producing less stress on the vertebral bodies and ensures a high degree of positional stability of the implant between the vertebrae.

### BACKGROUND OF THE INVENTION

The present invention relates to an implant for placement between vertebrae.

Implants for placement between vertebrae are used after resection of a vertebral disk to join the affected vertebrae by bridging the space now present between them. In this regard, the implant serves, on the one hand, as a spacer. The implant, which is provided with openings, also has a joining function, since it becomes penetrated by bony tissue, and the vertebrae become joined by the bony tissue in which the implant is embedded.

### SUMMARY OF THE INVENTION

The object of the present invention is to provide a new implant of the aforementioned type that is improved with respect to the functions mentioned above.

Pursuant to this object, and others which will become apparent hereafter, one aspect of the present invention resides in an implant having a shape adapted to a depression in the surfaces of the vertebrae that face the implant.

With respect to the shape of the implant, the invention takes into account the fact that the vertebral surfaces facing the implant are not flat, but rather are slightly depressed in the center. Surprisingly, this shape adaptation results in significant improvement of the positional stability of the implant between the vertebrae. Furthermore, strong compression of the surface and thus stress on the bony tissue by the implant are avoided. Both factors promote rapid, uncomplicated growth of the bony tissue connection with penetration of the implant.

In accordance with one embodiment of the invention, a shape adaptation of this type can consist in an increase in the height of the implant to a maximum height and then a decrease in height from the ventral side to the dorsal side of the spine. The maximum height preferably occurs in the last third of this longitudinal dimension of the implant, corresponding to the recessed profile of the surface of the vertebrae.

The implant offers an even more complementary opposing surface to the surface of the vertebrae, if, in addition, in the direction perpendicular to a center axis passing through the spine from front to back, the height of the implant increases towards the center axis. This shape adaptation takes into account the fact that the vertebral surface has approximately the shape of a slanted roof, with a ridge line that rises to a maximum and then falls off again.

The implant preferably conforms to the symmetry of the facing surfaces of the vertebrae by being symmetrically shaped with respect to a plane that perpendicularly intersects the longitudinal axis of the spine.

In an especially preferred embodiment of the invention, the implant is intended for placement in a half-space of the intervertebral space together with a second implant of this type, which shows mirror symmetry with respect to the first implant. Advantageously, each of these implants can be inserted into the intervertebral space by a dorsal approach through the vertebral canal that skirts the spinal cord. An implant of this type that would fill the entire intervertebral space could only be inserted by a ventral approach.

In a further embodiment of the invention, the implant has projections for fixing the implant in the bony tissue of the vertebrae. These projections contribute to further stabilization of the position of the implant between the vertebrae.

It is advantageous for these fixing projections to be positioned as far as possible from the vertebral canal and/or from the main load-bearing axis of the spine. In this way, when the fixing projections penetrate the bony tissue, there is no risk of either damaging nerve pathways or impairing the load-bearing capacity of the spine.

In a preferred embodiment of the invention, these fixing projections are designed as teeth.

In another advantageous embodiment of the invention, the front end face of the implant with respect to the direction of implantation has a convex surface to facilitate insertion of the implant into the intervertebral space. In a preferred embodiment of the invention, the implant has a cage-like design with openings in the walls.

In a further advantageous embodiment of the invention, the implant, as viewed from above, has a frame-like design with an through-opening in the frame that is open to the upper side and the lower side. Bony tissue can infiltrate the opening from both the upper side and the lower side of the implant to join the vertebrae. The openings in the sides of the implant may also be infiltrated by bony tissue, so that the implant becomes mostly embedded in bony tissue.

In a preferred embodiment of the invention, the implant consists of a plastic, preferably polyetheretherketone (PEEK). In contrast to metallic materials, plastic material is similar to bony tissue in its elasticity and therefore can become organically integrated in the bone matrix better than metal.

The various features of novelty, which characterize the invention, are pointed out with particularity in the claims annexed to and forming part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWINGS

In the drawings:

FIG. 1 shows a perspective view of an implant in accordance with the invention;

FIG. 2 shows a top view of the implant of FIG. 1.

FIG. 3 shows a longitudinal side view of the implant of FIG. 1;

FIG. 4 shows a front-end view of the implant of FIG. 1;

FIG. 5 shows a cross-sectional top view of the implant of FIG. 1;

FIG. 6 shows a cross-sectional front-end view of the implant of FIG. 1; and

FIG. 7 shows implants of the type illustrated in FIG. 1 in the implanted state between vertebrae.

### DETAILED DESCRIPTION OF THE INVENTION

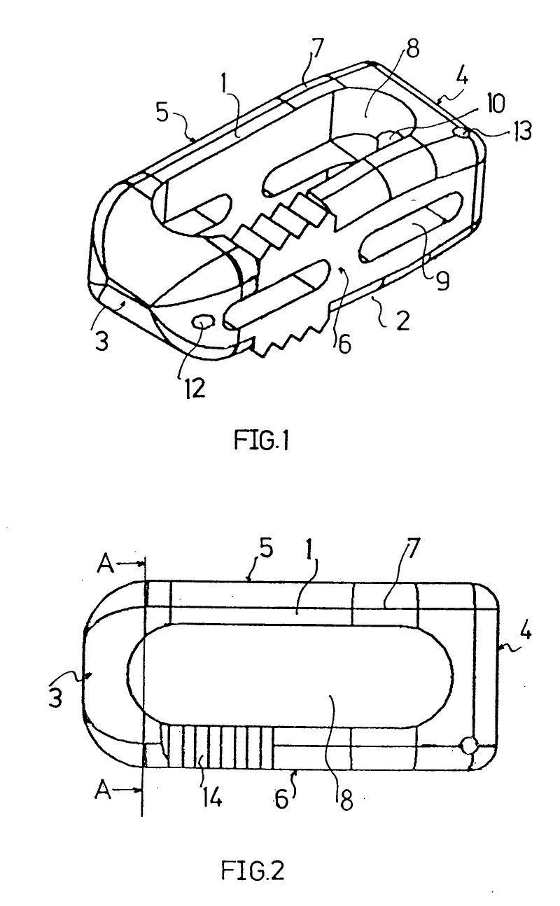

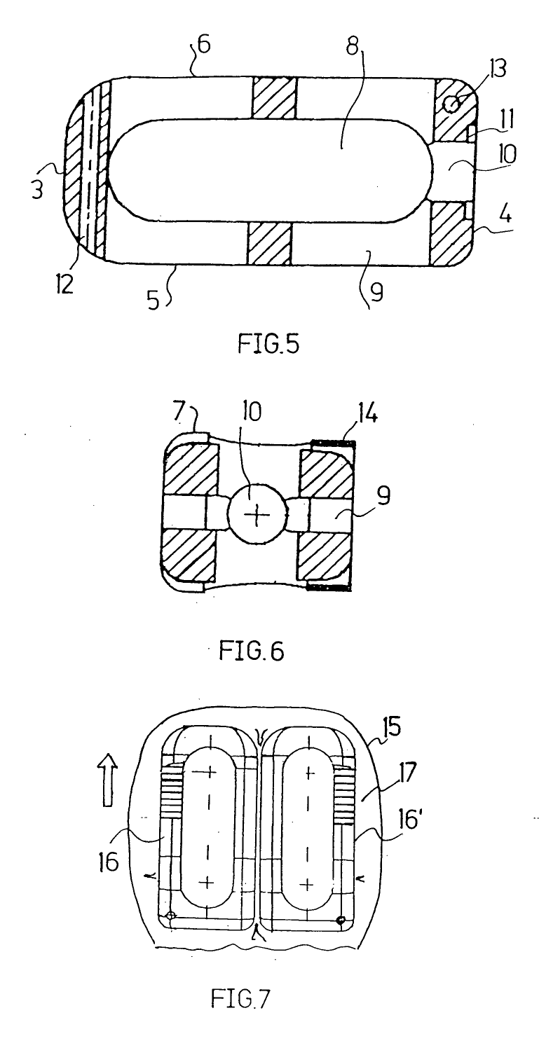

The implant shown in FIGS. 1 to 7 has the general contours of a rectangular solid with an upper side 1, a lower side 2, end faces 3 and 4, and longitudinal sides 5 and 6.

The rectangular solid has rounded edges and corners. The end face 3 is convex, while the opposite end face 4 is flat.

The shape of the implant deviates from a rectangular solid in that the height of the implant increases in the longitudinal direction from the end face 3, reaches a maximum at 7, and then, in the embodiment illustrated here, decreases to the end face 4. The maximum height of the implant at 7 is located in the last third of the distance from the end face 3 to the end face 4.

Finally, in another departure from the general form of a rectangular solid, the height of the implant decreases in the transverse direction from the longitudinal side 5 to the longitudinal side 6. In the embodiment illustrated here, this decrease in height is about 2 mm from the maximum height of the implant of 9.5 mm.

In the region from the end face 3 to the maximum height of the implant at 7, the upper side 1 and the lower side 2 of the implant are inclined toward each other by 6° with respect to the longitudinal axis of the implant. The corresponding inclination in the region between the maximum height of the implant and the end face 4 is 16°.

Accordingly, there is no symmetry with respect to vertical planes. However, the implant is symmetrical with respect to a horizontal plane intersecting the implant in the middle of its height.

As FIGS. 1 to 7 show, the implant has a vertical through-opening 8 that opens to the upper side 1 and the lower side 2, which gives the implant a frame-like appearance in the top views of FIGS. 2, 5, and 7, in which the vertical opening 8 represents the opening in the frame. As the drawings show, the vertical opening 8 has the shape of an oblong hole with rounded ends.

Each of the longitudinal sides 5,6 is provided with two openings 9, which open into the vertical opening 8 and, like the vertical opening 8, have the shape of an oblong hole with rounded ends.

A bore 10 that opens into the vertical opening 8 is formed in the end face 4. This bore 10 has a counterbore in the form of a slot 11 with slot segments extending diametrically with respect to the borehole.

Two bores 12,13 with mutually perpendicular axes are also formed in the implant. These bores are intended for holding metal pins, especially titanium pins, which are highly visible in radiographs.

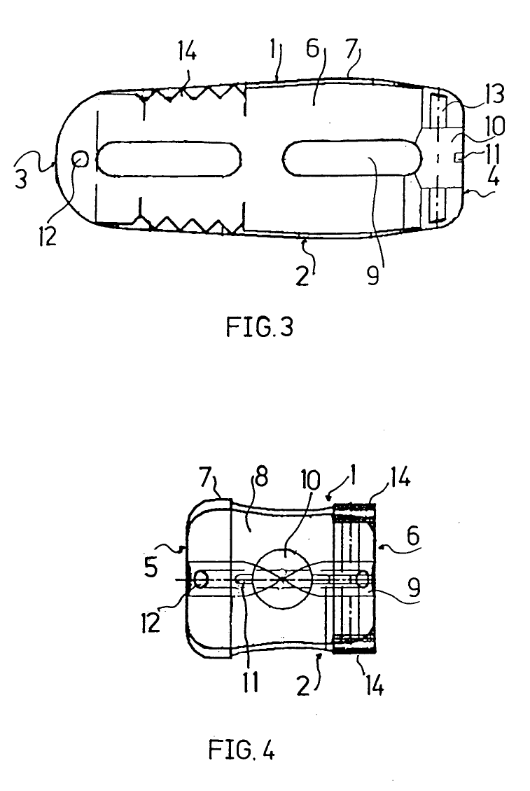

Teeth 14, which border the longitudinal side 6, are formed on the upper side 1 and the lower side 2 of the implant. The ridges of these teeth extend from the longitudinal side 6 to the vertical opening 8. As is shown especially by FIG. 3, the distance between the tooth ridges of corresponding teeth on the upper and the lower side of the implant corresponds to the implant height at corresponding points on the opposite longitudinal side 5.

The function of the implant described above will now be explained with reference to FIG. 7.

FIG. 7 shows two implants 16 and 16′ positioned between vertebrae 17 of the spine 15. Of these two implants, the implant 16 corresponds to the implant described above with reference to FIGS. 1 to 6. The implant 16′ is related to the implant 16 by mirror symmetry.

The implants are inserted into the intervertebral space between vertebral bodies in the direction of the arrow shown in FIG. 7. During the placement of the implant in the intervertebral space, the spinal cord in the vertebral canal (not shown) of the spine 15 is skirted. The convexity of the end face 3 facilitates the penetration of the implants into the space between the vertebrae, which are supported by suitable holders during the implantation to keep this space open.

To insert the implant 16 or 16′ into the intervertebral space, the implant can be moved by a tool, which engages the implant through the bore 10. The slot 11 forms a seat for the tool to secure the implant and tool against rotation with respect to each other.

In the position shown in FIG. 7, a scraping tool can be inserted through the bore 10 to allow removal of compact tissue from the bony surfaces of the vertebrae facing the implant in the region of the vertical opening. The parts of the implant surrounding the vertical opening then rest against compact tissue, while chiefly the exposed cancellous tissue forms new bony tissue, which can penetrate the vertical opening and ultimately leads to the joining of the two vertebrae adjacent to the implants 16 and 16′ by bony tissue. New growth of bony tissue can also penetrate the horizontal openings 9, so that the implant becomes largely embedded in the bony tissue joining the vertebrae.

The teeth 14 fix the implants 16, 16′ between the vertebrae of the spine 15 by penetrating the compact tissue. As viewed in the direction of insertion of the implants, the teeth 14 are located in the first half of the implant on the side of the implant facing the outside of the spine 15 and are thus located far from the spinal cord and far from the main load-bearing axis of the spine. Therefore, the penetration of the bony tissue by the teeth cannot impair either nerve pathways or the load-bearing capacity of the spine.

A special advantage of the implants 16, 16′ described above is their adaptation to the shape of the implant-side surfaces of the vertebrae to be joined. First, this shape is taken into account by the fact that the implant has a maximum height at 7. This maximum corresponds to a maximum of the depth of the vertebral surfaces at this point. Second, transversely to the direction of insertion of the implant, the depth of the vertebral surface decreases towards the sides. The decrease in the height of the implant from the longitudinal side 5 to the longitudinal side 6 takes this decrease in the depth of the vertebral surface into account.

The stress produced by the implant on the vertebrae to be joined is reduced by the substantial adaptation of the shape of the implant to the shape of the surface of the vertebral body. In addition, this shape adaptation contributes to the positional stability of the implant between the vertebrae. Both factors ultimately promote rapid growth of the bony tissue joining the vertebrae and thus the healing process after a diskectomy. This reduces the strength requirements for support devices necessary during the growth of the tissue joining the vertebrae.

In the embodiment shown here, the height of the implant at the end faces 3 and 4 is the same. In a departure from this configuration, the height at the end face 4 could be greater than at the end face 3, so that the overall shape of a blunt wedge would be obtained without the increase and decrease of the implant height between the end faces. Implants of this type with a basic wedge shape would be considered for use mainly for the lower lumbar region of the spine, where the vertebrae are normally inclined towards each other.

Instead of two implants of this type arranged side by side in an intervertebral space, it would also be possible to use a one-piece implant composed of the two implants, which slopes down to the lateral margins like a roof. In this case, however, only implantation by a front approach from the ventral side of the spine would be possible.

The position of the implants and thus their proper implantation can be checked on the basis of the radiographically determined position of the metal pins inserted in the bores 12 and 13.

The plastic used as the implant material, polyetherether-ketone (PEEK), has a high load-bearing capacity, and, in addition, has the advantage over metallic materials that it is similar in elasticity to bony tissue and can therefore become integrated in the bony tissue more readily than metal.

Depending on the size and use within the spine, the implant may have varying dimensions, especially in regard to the wedge angle and the maximum height.

Due to the great variety of implant variants that are necessary, the required quantity of implants with the same dimensions will remain small, so that machining is preferred over the injection molding process, although the latter can certainly be used to produce the implant, and mixed forms of machining with extensive prefabrication by injection molding are conceivable.

The invention is not limited by the embodiments described above which are presented as examples only but can be modified in various ways within the scope of the protection defined by the appended patent claims.

What is claimed is:

1. An implant for placement between vertebrae of a spine, wherein the implant has a shape adapted to a depression in vertebral surfaces facing the implant.

2. The implant in accordance with claim 1, wherein the implant has a height that increases from a ventral side to a dorsal side of the spine to a maximum height and then decreases again.

3. The implant in accordance with claim 2, wherein the maximum height, viewed in a direction from the ventral side to the dorsal side of the spine, is located in a last third of a length of the implant.

4. The implant in accordance with claim 1, wherein the implant has a height that increases towards the center axis in a direction perpendicular to a center axis passing through the spine from front to back.

5. The implant in accordance with claim 1, wherein the implant is symmetrically shaped with respect to a plane that perpendicularly intersects a longitudinal axis of the spine.

6. The implant in accordance with claim 1, wherein the implant is configured to be placeable in a half-space of an intervertebral space together with another implant having with mirror symmetry with respect to the first implant.

7. The implant in accordance with claim 1, wherein the implant has projections arranged and configured fix the implant in the bony tissue of the vertebrae.

8. The implant in accordance with claim 1, wherein the implant has an anterior end face, with respect to a direction of implantation, that has a convex curvature towards the front.

9. The implant in accordance with claim 1, wherein the implant has a hollow, cage-like configuration with wall openings.

10. The implant in accordance with claim 1, wherein the implant, as viewed from above, has a frame-like configuration with an opening in the frame that is open to an upper side and a lower side.

11. The implant in accordance with claim 1, wherein the implant consists of a plastic preferably PEEK.

12. The implant in accordance with claim 11, wherein the implant consists of PEEK.
